(19) United States
(12) Patent Application Publication
(10) Pub. No.: US 2020/0317608 A1
(43) Pub. Date: Oct. 8, 2020

(54) METHOD AND SYSTEM COMBINATION FOR THE PREPARATION OF UREA

(71) Applicant: LINDE AKTIENGESELLSCHAFT, Munchen (DE)

(72) Inventors: HELMUT FRITZ, Munich (DE); Ernst HAIDEGGER, Riemerling (DE); Rainer KEMPER, Munich (DE); Heinz ZIMMERMANN, Munich (DE)

(21) Appl. No.: 16/303,842

(22) PCT Filed: May 12, 2017
(86) PCT No.: PCT/EP2017/061498

§ 371 Date: Nov. 21, 2018

(30) Foreign Application Priority Data
May 24, 2016 (EP) 16171095.9

(51) Int. Cl. C07C 273/04; C07C 2/82; C01B 32/50; C01B 3/02; C01C 1/04; C07D 301/08; B01J 19/24; F25J 3/04

(52) U.S. Cl. CPC C07C 273/04; C07C 2/82; C01B 32/50; C01B 3/025; B01J 2219/0004; C07D 301/08; B01J 19/245; F25J 3/04484; C01C 1/0488

(57) ABSTRACT

The invention relates to a process (100), in which, with the inclusion of an air-separation method (10), an oxygen-rich substance flow (b) is formed, which is subjected with a methane-rich substance flow (e) to a method for oxidative coupling of methane (20). From a product flow (e) of the method for oxidative coupling of methane (20), a carbon-dioxide-rich substance flow (i) is formed and subjected to a urea-synthesis method (50). A corresponding combined plant also forms the subject matter of the invention.

The invention relates to a process for the manufacture of urea and a corresponding combined plant according to the preambles of the respective independent claims.

### PRIOR ART

Methane, for example, from natural gas, is currently used predominantly for burning. However, an alternative substance use is of great interest from a commercial perspective. For example, methods for the manufacture of higher hydrocarbons from methane through oxidative coupling of methane (English: Oxidative Coupling of Methane, OCM) are currently being intensively developed. Oxidative coupling of methane refers to the direct conversion of methane in an oxidative, heterogeneously catalysed method to form higher hydrocarbons. Corresponding methods are used especially for the manufacture of ethylene.

For further details of oxidative coupling of methane, reference is made to the relevant specialist literature, for example, Zavyalova, et al.: Statistical Analysis of Past Catalytic Data on Oxidative Methane Coupling for New Insights into the Composition of High-Performance Catalysts, ChemCatChem 3, 2011, 1935-1947.

In the oxidative coupling of methane, a methane-rich substance flow, for example, natural gas or a substance flow formed from natural gas, is supplied to a reactor together with an oxygen-rich substance flow. A product flow is formed, which contains, alongside reaction products of the oxidative coupling of methane, especially ethylene, optionally propylene, hydrogen, carbon dioxide, unconverted methane and unconverted oxygen. If, for example, nitrogen-containing natural gas is used, the product flow will also contain nitrogen.

The oxygen-rich substance flow used for the oxidative coupling of methane is typically supplied through an air-separation method. The manufacture of air products by means of corresponding air-separation methods has been known for a considerable time and is described, for example, in H.-W. Häring (Ed.), Industrial Gases Processing, Wiley-VCH, 2006, especially Subsection 2.2.5, “Cryogenic Rectification”. The present invention accordingly relates especially to such air-separation methods which are used for the generation of gaseous, oxygen-rich substance flows.

In principle, there is a need to improve the exploitation of products from the oxidative coupling of methane and to increase the overall yield from corresponding processes.

### DISCLOSURE OF THE INVENTION

This object is achieved by a process for the manufacture of urea and a corresponding combined plant with the features of the independent claims. In each case, further developments form the subject matter of the dependent claims and of the subsequent description.

Liquid and gaseous substance flows can be described in the conventional linguistic usage in this context as rich or poor in one or more components, wherein the term “rich” denotes a content of at least 50%, 75%, 90%, 95%, 99%, 99.5%, 99.9% or 99.99% and the term “poor” denotes a maximum content of 50%, 25%, 10%, 5%, 1%, 0.1% or 0.01% on a molar, weight or volume basis.

If a substance flow is formed, in the conventional linguistic usage here, “with the inclusion” of a given method, for example, of an air-separation method or a method for oxidative coupling of methane, this explicitly does not exclude the participation of other methods, especially separation methods, from the formation of the substance flow. Similarly, the formulation does not exclude the formation of additional substance flows of respectively the same or different composition through corresponding methods.

### Advantages of the Invention

Against the background explained above, the present invention proposes a process in which, with the inclusion of an air-separation method, an oxygen-rich substance flow is formed, which is subjected, with a methane-rich substance flow, to a method for oxidative coupling of methane. To this extent, the process according to the invention does not differ from the processes of the prior art explained in the introduction. However, within such a process, the invention additionally proposes the formation, from the product flow of the method for oxidative coupling of methane, of a carbon-dioxide-rich substance flow, which is subjected to a synthesis method for the production of urea.

As has been shown within the scope of the present invention, the coupling of the oxidative coupling of methane with a corresponding synthesis method, as proposed according to the invention, is particularly suitable for increasing the overall efficiency of corresponding processes.

In a product flow of a method for oxidative coupling of methane, carbon dioxide is typically present in not insignificant quantities. From a conventional perspective, the carbon dioxide is an undesirable by-product. As explained, for example, by Zavyalova et al. (see above), a non-selective oxidation of the methane and the hydrocarbons formed occurs in the oxidative coupling of methane to give carbon monoxide and carbon dioxide. Conventionally, appropriate catalysts for the oxidative coupling of methane should therefore not only catalyse the formation of methyl radicals, which then react to form ethane and ethylene, but also suppress the non-selective oxidation of the methane and the hydrocarbons formed. If a method according to the invention is used, carbon dioxide can be converted in its entirety to form products, so that this aspect has a reduced significance and increased freedoms are achieved in the optimisation of a corresponding catalyst.

Corresponding carbon dioxide is typically removed from corresponding product flows upstream of a separation method, to prevent freezing out and accordingly displacement of plant components at the separating temperatures and pressures used. For the separation of carbon dioxide, a per se known carbon-dioxide wash or scrubbing is typically used. The carbon dioxide obtained in this context is particularly suitable for use in a urea-synthesis method, as utilised according to the invention. For the manufacture of the ammonia required in such a urea-synthesis method, a series of further compounds occurring in a corresponding process can also be used.

In this context, it is particularly advantageous if an ammonia-synthesis method is initially performed and the latter is also integrated into the process according to the invention. Ammonia, which is formed in a corresponding ammonia-synthesis method, can then be converted with the carbon dioxide from the product flow of the method for oxidative coupling of methane to form urea, as provided according to the invention.

In the process variant proposed, the process allows, for example, a further integration of air separation and oxidative coupling of methane in that nitrogen formed in the air separation, which is not used in the oxidative coupling of methane, is subjected to the ammonia-synthesis method. However, nitrogen for the ammonia-synthesis method can also originate completely or partially from the product flow of the method for oxidative coupling of methane.

Hydrogen required in the ammonia-synthesis method can also originate from the product flow of the method for oxidative coupling of methane and/or from another method or respectively another source. For example, methane or natural gas which is also provided for the method for oxidative coupling of methane can be subjected in parallel to a hydrogen synthesis method of known type, for example, a steam reforming. Hydrogen formed in this manner can be used alone or together with hydrogen which is contained in the product flow of the method for oxidative coupling of methane. Accordingly, for example, an inadequate or fluctuating hydrogen content in the product flow of the method for oxidative coupling of methane can be compensated. The preferred source for hydrogen is also determined by its accessibility. For example, if a recovery of hydrogen from the product flow of the method for oxidative coupling of methane proves too effort intensive, it is also possible to draw exclusively on hydrogen which is obtained by means of a separate hydrogen-synthesis method of the type explained.

According to the advantageous embodiment just explained, the process can therefore use a nitrogen-rich substance flow formed with the inclusion of the air-separation method. As an alternative or additionally, it is possible to use nitrogen which is contained in the product flow of the method for oxidative coupling of methane.

From this nitrogen, in this context, initially together with hydrogen, ammonia can also be synthesised, which is then subjected to the synthesis method for the production of urea, together with the carbon-dioxide-rich substance flow. If a nitrogen-rich substance flow formed with the inclusion of the air-separation method is used for the production of the ammonia, arbitrary quantities of nitrogen can be provided, so that the process is completely independent of any nitrogen contained, possibly only in small proportions or not at all, in the waste flow of the method for oxidative coupling of methane. A corresponding process variant is therefore especially suitable for cases in which a product flow of the method for oxidative coupling of methane comprises no nitrogen content or an insufficient nitrogen content, or for the compensation of fluctuations in its nitrogen content.

In fact, temperatures and pressures such as are used in synthesis methods for the production of ammonia, are, in some cases, disposed significantly above those used in the oxidative coupling of methane. However, the process according to the embodiment of the invention just explained provides special advantages if, in a corresponding synthesis method for the production of ammonia, the nitrogen which is contained in the product flow of the method for oxidative coupling of methane is used. In this case, no compression starting from atmospheric pressure and/or no temperature increase starting from ambient temperature, or optionally below, is necessary, as might be required with the use of nitrogen from the air-separation method. The energy to be expended is therefore significantly reduced.

Synthesis methods for the production of ammonia and urea are known in principle. For details of both methods, reference is made to the specialist literature, for example, the article “Ethylene” mentioned in Ullmann's Encyclopedia of Industrial Chemistry, Online Publication 15 Dec. 2006, doi:10.1002/14356007.a02_143.pub2, and the article “Urea” in Ullmann's Encyclopedia of Industrial Chemistry, Online Publication 15 Jun. 2000, doi:10.1002/14356007.a27_333.pub2.

As already mentioned, the hydrogen contained in the product flow of the method for oxidative coupling of methane can be subjected to an ammonia-synthesis method. The present invention accordingly allows an advantageous use of the hydrogen formed in the oxidative coupling of methane.

In particular, this process variant achieves special advantages in cases in which the product flow of the method for oxidative coupling of methane contains nitrogen, because this nitrogen need not then be separated from the hydrogen. A corresponding product flow may contain nitrogen for different reasons, wherein the process according to the invention is suitable in all cases.

Accordingly, the present process proves advantageous, especially in cases in which the methane-rich substance flow which is subjected to the method for oxidative coupling of methane does not comprise only small quantities of nitrogen. Since this nitrogen is typically hardly converted or not converted at all in the method for oxidative coupling of methane, it is transferred into the product flow and must conventionally be separated in an effort-intensive manner. With a boiling point of −196° C. (nitrogen) and −252° C. (hydrogen), nitrogen and hydrogen represent the components with the lowest boiling points in corresponding product flows. The other compounds contained in significant quantities in corresponding product flows boil at significantly higher temperatures. A separation of hydrogen and nitrogen would accordingly require, for example, an effort-intensive low-temperature separation or a membrane process, which is disadvantageous for commercial reasons and/or would require effort-intensive additional separation equipment. The same also applies for a recovery of nitrogen from natural gas, which would have to take place in upstream method steps.

However, if a hydrogen-rich substance flow formed from a corresponding product flow is subjected to an ammonia-synthesis method, any nitrogen contained is not problematic here. In this context, if the quantity of nitrogen contained in the product flow of the method for oxidative coupling of methane is not sufficient for the stoichiometric conversion with hydrogen, a further nitrogen-rich substance flow can be used within the scope of the embodiment of the invention just explained, which can be formed, as explained previously, with the inclusion of the air-separation method.

If nitrogen-containing, methane-rich substance flows are used as feedstock for the oxidative coupling of methane, these can comprise, for example, a nitrogen content of up to 20 mole percent, especially from 1 to 5 or 5 to 10 mole percent, wherein nitrogen contained in the methane-rich substance flow is transferred completely into the hydrogen-rich substance flow and, subjected to the ammonia-synthesis method within the latter. As explained previously, in this process variant, the present invention dispenses with a nitrogen recovery from the natural gas and/or a separation of hydrogen and nitrogen in a corresponding product flow.

However, in the embodiment explained, the present invention also allows the use of a more energy-efficient air-separation method, because pure oxygen in the form of the oxygen-rich substance flow need not necessarily be supplied to the method for oxidative coupling of methane. Accordingly, a less rigid separation of nitrogen and oxygen can be implemented. As explained, in corresponding methods for oxidative coupling of methane, nitrogen is hardly converted or not converted at all, so that the latter is transferred into a corresponding product flow. The nitrogen contained in the product flow can then be used in an ammonia-synthesis method as explained. Accordingly, the present invention also allows the use of oxygen-rich substance flows with a content of, for example, up to 20 mole percent, especially of 1 to 5 or 5 to 10 mole percent, nitrogen. For example, the invention allows the use of air-separation plants with mixing columns. Corresponding plants and methods have been disclosed many times elsewhere, for example, in EP 1 139 046 B1. For details of the optimisation of air-separation plants, reference is made to the relevant specialist literature, for example, Section 3.8 in Kerry, F. G., Industrial Gas Handbook: Gas Separation and Purification, Boca Raton: CRC Press, 2006.

However, the present invention allows an even greater integration of the named process components and respectively methods. Accordingly, it is particularly advantageous if, from the product flow of the method for oxidative coupling of methane, one or more olefin-rich substance flows and, with the inclusion of the air-separation method, one or more further oxygen-rich substance flows are also formed. The olefin-rich substance flow or flows and the further oxygen-rich substance flow or flows can be subjected, together, to an epoxidation method. Corresponding epoxidation methods can be provided separately for an olefin-rich substance flow and or for several in combination. In particular, only one olefin-rich substance flow may be epoxidised. One or more corresponding olefin-rich substance flows are rich in ethylene and/or propylene. With a corresponding epoxidation, propylene oxide is formed from propylene and ethylene oxide is formed from ethylene, that is, compounds which are particularly suitable as starting components for further reactions. In particular, it can be provided to synthesise ethylene glycol and/or propylene glycol from ethylene oxide and/or propylene oxide formed in the epoxidation method.

The present invention also allows the recycling of substance flows, for example, in that at least one further substance flow is formed from the product flow of the method for oxidative coupling of methane, which is again subjected to the method for oxidative coupling of methane. The at least one further substance flow which, in particular, can contain methane is, advantageously in this context, poor in nitrogen or free from nitrogen, however, it can contain nitrogen, if nitrogen is drawn continuously from a corresponding circulation, within the framework of the method according to the invention, that is, for example, supplied to the ammonia-synthesis method.

The present invention relates further to a combined plant, which comprises an air-separation plant and at least one reactor equipped for the implementation of a method for the oxidative coupling of methane. The plant complex comprises means, which are equipped, with the inclusion of an air-separation method implemented in the air-separation plant, to form an oxygen-rich substance flow and to subject the latter, with a methane-rich substance flow, to a method for oxidative coupling of methane in the at least one reactor. According to the invention, means are provided which are equipped to form, from the product flow of the method for oxidative coupling of methane, a carbon-dioxide-rich substance flow and to subject the latter to a urea-synthesis method in one or more further reactors.

A corresponding combined plant is advantageously equipped for the implementation of a method as was explained previously and provides corresponding means for this purpose. Regarding features and advantages of the corresponding plant complex, explicit reference is therefore made to the above explanations.

The invention is explained in greater detail below with reference to the attached drawing which shows a preferred embodiment of the invention.

### BRIEF DESCRIPTION OF THE DRAWING

FIG. 1 shows a process for manufacturing reaction products according to a particularly preferred embodiment of the invention.

### DETAILED DESCRIPTION OF THE DRAWING

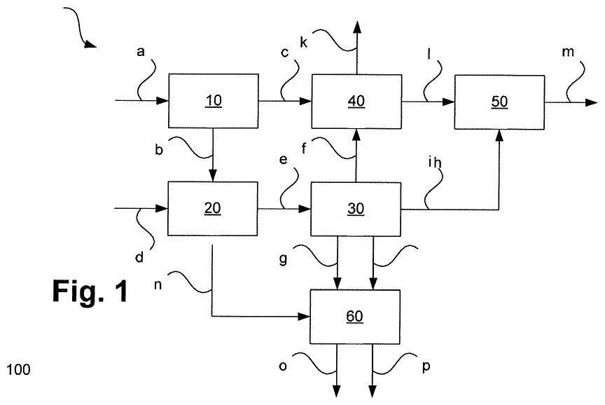

In FIG. 1, a process according to a particularly preferred embodiment of the invention is shown in the form of a schematic process-flow diagram and marked as a whole with 100.

The process 100 comprises an air-separation method 10 and a method for oxidative coupling of methane 20. Input air in the form of a substance flow a is supplied to the air-separation method 10. Air-separation methods 10 suitable for use within the scope of the process 100 have been described extensively elsewhere.

With the use of a corresponding air-separation method 10 in the illustrated example, an oxygen-rich substance flow b and a nitrogen-rich substance flow c are prepared. However, arbitrary further substance flows, which can comprise air-separation products, can also be provided with the use of the air-separation method 10, for example, further oxygen-rich and/or nitrogen-rich substance flows and/or substance flows which are rich in one or more noble gases, as is known in principle.

In the illustrated example, the oxygen-rich substance flow b and a methane-rich substance flow d, which can be, for example, conditioned or non-conditioned natural gas, are supplied to the method for oxidative coupling of methane 20. In the method for oxidative coupling of methane 20, a product flow e is generated, which can contain, inter alia, unconverted methane of the substance flow d, unconverted oxygen of the substance flow b, inert gases such as nitrogen optionally contained in the substance flow d, and reaction products of the oxidative coupling of methane, such as hydrogen, carbon dioxide, ethylene or propylene.

The product flow e is subjected to a separation method 30, which can comprise non-cryogenic and cryogenic separation steps. In particular, the separation method 30 can also comprise a gas scrubbing. Especially a hydrogen-rich substance flow f, an ethylene-rich substance flow g, a propylene-rich substance flow h and a carbon-dioxide rich substance flow i can be provided with the use of the separation method 30. The hydrogen-rich substance flow f, the propylene-rich substance flow g and the ethylene-rich substance flow h are typically produced in one or more cryogenic separation steps of the separation method 30. The carbon-dioxide-rich substance flow i is typically separated in advance. In the separation method 30 or respectively in corresponding separation steps, further substance flows can also be provided, which have, however, not been shown in FIG. 1 for the sake of visual clarity.

In the embodiment shown in FIG. 1, the implementation of an ammonia-synthesis method 40 takes place, to which, the nitrogen-rich substance flow c, which is prepared with the use of the air-separation method 10, and the hydrogen-rich substance flow f, which is prepared with the use of the method for oxidative coupling of methane and the downstream separation method 30, are supplied in the illustrated example within the framework of the process 100. However, as mentioned several times, a corresponding hydrogen-rich substance flow f can also originate from other sources, for example, from a steam reforming method. In principle, ammonia can also originate from different sources.

It should be emphasised that, with the use of the method for oxidative coupling of methane 20 or respectively of the downstream separation method 30, further hydrogen-rich flows can also be provided, which need not necessarily be supplied in their entirety to the ammonia-synthesis method 40. Similarly, the nitrogen supplied to the ammonia-synthesis method 40 need not originate or need not originate exclusively from the nitrogen-rich substance flow c from the air-separation method 10. At least a part of the nitrogen can also be contained in the hydrogen-rich substance flow f, as explained above, especially if the latter originates from a method for oxidative coupling of methane.

With the use of the ammonia-synthesis method 40, two ammonia-rich flows k and l are provided in the illustrated example. The particularly preferred embodiment of the process 10 illustrated in FIG. 1 comprises a urea-synthesis method 50. In this context, the ammonia-rich flow l, which is prepared with the use of the ammonia-synthesis method 40, and the carbon-dioxide-rich flow i, which is prepared with the use of the method for oxidative coupling of methane 20 and the downstream separation method 30, are supplied to the urea-synthesis method 50. It goes without saying that the entire ammonia formed in the ammonia-synthesis step 40 and/or the entire carbon dioxide provided in the method for oxidative coupling of methane 20 and the downstream separation method 30 need not be supplied to the urea-synthesis method 50. In each case, only partial quantities of the named compounds can also be used; the remainder can be output from a corresponding process 100, for example, as a product or respectively by-product. A corresponding case is shown in FIG. 1 with the ammonia-rich substance flows k and l.

In the illustrated example, the ammonia-rich substance flow k is output from the process. With the use of the urea-synthesis method 50 in the particularly preferred embodiment of the invention illustrated in FIG. 1, a urea-rich substance flow m is provided and supplied as required to appropriate conditioning steps.

The methods explained in the following are also not necessarily a component of a corresponding process 100. This means that the propylene-rich substance flow g and/or the ethylene-rich substance flow h can also, in each case, be output as products from a corresponding process 100.

The illustrated example shows an epoxidation method 60 which can also be provided separately for the propylene-rich substance flow g and the ethylene-rich substance flow h or only for one of these substance flows. Furthermore, an oxygen-rich substance flow n, which can, in particular, be provided with the use of the air-separation method 10, is supplied to the epoxidation method 60. With the use of the epoxidation method 60, a propylene-oxide-rich substance flow o and/or an ethylene-oxide-rich substance flow p can be provided. Here also, the entire propylene and/or ethylene provided in the method for oxidative coupling of methane 20 or respectively the downstream separation method need not be subjected to the epoxidation method 60. In particular, partial flows of corresponding propylene or respectively ethylene can be output as products from the process 100.

1. A process, in which, with the inclusion of an air-separation method, an oxygen-rich substance flow is formed, which is subjected, with a methane-rich substance flow, to a method for oxidative coupling of methane, characterised in that a carbon-dioxide-rich substance flow is formed from a product flow of the method for oxidative coupling of methane and subjected to a urea-synthesis method.

2. The process according to claim 1, in which, with the inclusion of an air-separation method, a nitrogen-rich substance flow is further formed and subjected to an ammonia-synthesis method.

3. The process according to claim 2, in which, from the product flow, a hydrogen-rich substance flow is further formed and subjected to the ammonia-synthesis method.

4. The process according to claim 3, in which the methane-rich substance flow contains nitrogen, wherein the nitrogen contained in the methane-rich substance flow is partially or completely transferred into the hydrogen-rich substance flow and subjected to the ammonia-synthesis method within the latter.

5. The process according to claim 4, in which the methane-rich substance flow contains up to 20 mole percent nitrogen.

6. The process according to claim 3, in which the oxygen-rich substance flow contains nitrogen, wherein the nitrogen contained in the oxygen-rich substance flow is partially or completely transferred into the hydrogen-rich substance flow and subjected to the ammonia-synthesis method within the latter.

7. The process according to claim 6, in which the oxygen-rich substance flow contains up to 20 mole percent nitrogen.

8. The process according to claim 1, in which, from the product flow, one or more olefin-rich substance flows are further formed, and, with the inclusion of an air-separation method, one or more further oxygen-rich substance flows are formed, wherein the olefin-rich substance flow or flows and the further oxygen-rich substance flow or flows are subjected to an epoxidation method.

9. The process according to claim 1, in which, from the product flow, at least one further substance flow is formed, which is again subjected to the method for oxidative coupling of methane.

10. The process according to claim 1, in which the waste heat of the method for oxidative coupling of methane is used for the pre-heating or heating of one or more substance flows and/or of one or more reactors, which are used in the synthesis method for the production of the nitrogen-containing synthesis product or products.

11. A combined plant which comprises an air-separation plant and at least one reactor equipped for the implementation of a method for oxidative coupling of methane, wherein the combined plant comprises means, which are equipped, with the inclusion of an air-separation method implemented in the air-separation plant, to form an oxygen-rich substance flow and to subject the latter, with a methane-rich substance flow, to a method for oxidative coupling of methane in the at least one reactor, characterised in that means are provided which are equipped to form a carbon-dioxide-rich substance flow from a product flow of the method for oxidative coupling of methane and to subject it to a urea-synthesis method.

12. The combined plant according to claim 11, which is equipped to implement a method comprising a process in which, with the inclusion of an air-separation method, an oxygen-rich substance flow is formed, which is subjected, with a methane-rich substance flow, to a method for oxidative coupling of methane, characterised in that a carbon-dioxide-rich substance flow is formed from a product flow of the method for oxidative coupling of methane and subjected to a urea-synthesis method.

13. The process according to claim 5, in which the methane-rich substance flow contains from 5 to 10 mole percent nitrogen.

14. The process according to claim 7, in which the oxygen-rich substance flow contains from 5 to 10 mole percent nitrogen.
